(19) United States
(12) Patent Application Publication
(10) Pub. No.: US 2024/0034970 A1
(43) Pub. Date: Feb. 1, 2024

(54) CELL CULTURE SUBSTRATE AND MANUFACTURING METHOD THEREOF

(71) Applicants: Yusuke Nonoyama, Kanagawa (JP); Takahiko Matsumoto, Kanagawa (JP); Daisuke Arai, Tokyo (JP)

(72) Inventors: Yusuke Nonoyama, Kanagawa (JP); Takahiko Matsumoto, Kanagawa (JP); Daisuke Arai, Tokyo (JP)

(73) Assignee: Ricoh Company, Ltd., Tokyo (JP)

(21) Appl. No.: 18/360,318

(22) Filed: Jul. 27, 2023

(30) Foreign Application Priority Data
Jul. 29, 2022 (JP) 2022-121195

(51) Int. Cl. C12M 1/32; C12M 1/12

(52) U.S. Cl. CPC C12M 23/12; C12M 25/16

(57) ABSTRACT

A method for manufacturing a cell culture substrate in which cells are fixed via a hydrogel on a surface of a substrate includes forming a film of a first liquid on the surface of the substrate, and ejecting droplets of a second liquid onto the film.

### BACKGROUND OF THE INVENTION

### Field of the Invention

The present invention relates to a cell culture substrate and a manufacturing method thereof.

### Description of Related Art

In recent years, demand has increased for toxicity and drug efficacy evaluation tools for use in vitro. One of the reasons for the foregoing is the promotion of the three R's (“Replacement,” “Reduction,” and “Refinement”) regarding animal testing, which has led to demand for a reduction in the number of test animals, alternative methods to replace test animals, and the like. As reasons other than the promotion of the three R's for animal testing, toxicity and drug efficacy evaluation tools for use in vitro have many advantages, such as reducing the cost of animal testing and reducing testing time.

On the other hand, along with advances in tissue engineering, techniques are being developed to artificially form tissues formed of a plurality of cells. Among the above, bioprinters have attracted attention in the construction of three-dimensional tissue models since it is possible to quickly fix cells to the bottom surface of a culture container or on gels, depending on the type of fixative. However, in vitro, it is difficult to completely reproduce the function of cells and the process of reaction to drugs in vivo and no such methods have yet been established.

Therefore, as one method of constructing a tissue model in vitro, Patent Document 1 discloses a method for manufacturing a substrate for evaluation, which enables cells to adhere to the substrate. Specifically, Patent Document 1 discloses a method for manufacturing a substrate for evaluation including a fixative A-containing solution-imparting step for imparting a solution containing a fixative A to a substrate, and a fixative B and cells-containing suspension imparting step for imparting a suspension containing a fixative B and cells, which is able to be mixed with the fixative A, in which at least a part of each of the fixative A and the fixative B are mixed to form particles of hydrogel, the cells are fixed to the substrate via the hydrogel, and the suspension is imparted as droplets such that the cells are arranged without overlapping. It is already known that the method described in Patent Document 1 makes it possible to create patterns with hydrogel particles including cells and that this method is an important means of constructing tissue models in vitro.

### SUMMARY OF THE INVENTION

However, the inventors found a problem in that it is difficult to uniformly coat the fixative A-containing solution at a desired thickness in the method described in Patent Document 1. In particular, in a case of using a multi-hole plate such as a well plate, which is frequently used in drug discovery research, when a fixative A-containing solution with low viscosity and high surface tension, such as with water as the main component, is coated on the bottom surface of the wells, the fixative A-containing solution is attracted to the wall surfaces of the wells due to surface tension such that it is difficult to uniformly coat the fixative A-containing solution at the desired thickness. Due to this, there are concerns that the shape of the hydrogel particles may become non-uniform, cells may not be encapsulated in the hydrogel particles and may leak out, the shape of the hydrogel particles may become flat, the hydrogel particles may not be able to sufficiently contain moisture, causing damage to the cells due to drying, and, in the worst case, cell death may occur.

The present invention was made in view of the above circumstances and provides a method for manufacturing a cell culture substrate with which it is possible to uniformly form a liquid film at a desired thickness and obtain uniformly shaped hydrogel particles including cells, as well as a cell culture substrate obtained by this manufacturing method.

The method for manufacturing a cell culture substrate is a method for manufacturing a cell culture substrate in which cells are fixed via a hydrogel on a surface of a substrate, the method including a step for forming a film of a first liquid on the surface of the substrate, and a step for ejecting droplets of a second liquid onto the film, in which the hydrogel has a polymer formed by bonding a first fixative and a second fixative, and water included in the polymer, the first liquid is an aqueous solution including the first fixative, the second liquid is a suspension including the second fixative and the cells, a recess is formed in the substrate at a position where the film is to be formed and the film fills an interior of the recess, at least parts of the first liquid forming the film and the second liquid forming the droplets are mixed to form the particles of hydrogel corresponding to a size of the droplets in the step for ejecting the droplets, and the particles encapsulate the cells and are attached to the surface of the substrate.

### Effect of the Invention

According to the method for manufacturing a cell culture substrate in the aspect described above, it is possible to uniformly form a liquid film at a desired thickness and to obtain uniformly shaped hydrogel particles including cells.

The cell culture substrate of the aspect described above is obtained by this manufacturing method and has uniformly shaped hydrogel particles including cells.

### BRIEF DESCRIPTION OF THE DRAWINGS

FIG. 1A is a plan view of a cell culture substrate according to a first embodiment of the present invention.

FIG. 1B is a cross-sectional view of the cell culture substrate cut along a plane passing through an I-I′ line shown in FIG. 1A.

FIG. 2A is a plan view of a cell culture substrate according to a second embodiment of the present invention.

FIG. 2B is a cross-sectional view of the cell culture substrate cut along a plane passing through an II-II′ line shown in FIG. 2A.

FIG. 3A is a plan view of a cell culture substrate according to a third embodiment of the present invention.

FIG. 3B is a cross-sectional view of the cell culture substrate cut along a plane passing through an line shown in FIG. 3A.

FIG. 4A is a plan view of a cell culture substrate according to a fourth embodiment of the present invention.

FIG. 4B is a cross-sectional view of the cell culture substrate cut along a plane passing through an IV-IV′ line shown in FIG. 4A.

FIG. 5A is a plan view of a cell culture substrate according to a fifth embodiment of the present invention.

FIG. 5B is a cross-sectional view of the cell culture substrate cut along a plane passing through a V-V′ line shown in FIG. 5A.

FIG. 6A is a plan view of a cell culture substrate according to a sixth embodiment of the present invention.

FIG. 6B is a cross-sectional view of the cell culture substrate cut along a plane passing through a VI-VI′ line shown in FIG. 6A.

FIG. 7 is a schematic diagram showing an example of a piezoelectric ejecting head used in a method for manufacturing a cell culture substrate according to an embodiment of the present invention.

FIG. 8 is a schematic diagram showing an example of an apparatus used in the method for manufacturing a cell culture substrate according to an embodiment of the present invention.

FIG. 9 is a schematic diagram showing a modified example of the apparatus used in the method for manufacturing a cell culture substrate according to an embodiment of the present invention.

FIG. 10 is a schematic diagram showing a modified example of the apparatus used in the method for manufacturing a cell culture substrate according to an embodiment of the present invention.

FIG. 11 is a diagram illustrating a hardware block of a control means of FIG. 8.

FIG. 12 is a schematic diagram showing processes of the method for manufacturing a cell culture substrate according to an embodiment of the present invention.

FIG. 13 is bright field images (top) of a film of the first liquid and cross-sectional views (bottom) of wells in a case where a method for manufacturing a cell culture substrate of the related art is applied to a 96-well plate.

FIG. 14 is a schematic diagram showing processes of the method for manufacturing a cell culture substrate of the related art.

FIG. 15 is microscopic images of hydrogel particles on cell culture substrates manufactured in Example 1 and Comparative Example 1.

FIG. 16 is microscopic images of hydrogel particles on the cell culture substrate manufactured in Example 1 immediately after manufacturing (day 0) and 72 hours thereafter (day 3).

### DETAILED DESCRIPTION OF THE INVENTION

A description will be given below of an embodiment of the present invention (may be simply referred to below as “the present embodiment”) with reference to specific embodiments and drawings as necessary. Such embodiments and drawings are only examples to facilitate understanding of the present invention and do not limit the present invention. That is, it is possible to change or improve the shape, dimensions, arrangements, and the like of the members described below without departing from the gist of the present invention and the present invention includes equivalents thereof. In addition, in all the drawings, similar components will be marked with similar reference numerals and overlapping explanations thereof will not be included as appropriate.

Unless separately defined in the present specification, all technical terms and scientific terms used in the present specification have the same meanings as those ordinarily understood by a person skilled in the art. All patents, applications, other publications, and information referred to in the present specification are incorporated in the present specification by reference in the entireties thereof. In addition, in a case of any inconsistency between the publications referred to in the present specification and the description in the present specification, the description in the present specification takes priority.

<Method For Manufacturing Cell Culture Substrate>

The method for manufacturing a cell culture substrate of the present embodiment is a method for manufacturing a cell culture substrate in which cells are fixed via a hydrogel on a surface of a substrate, in which the following steps are included.

A step for forming a film of a first liquid on the surface of the substrate (may be referred to below as the “film forming step”); and A step for ejecting droplets of a second liquid onto the film (may be referred to below as the “droplet ejecting step”).

In the method for manufacturing a cell culture substrate of the present embodiment, the hydrogel has a polymer formed by bonding a first fixative and a second fixative, and water included in the polymer.

In the method for manufacturing a cell culture substrate of the present embodiment, the first liquid is an aqueous solution including the first fixative.

In the method for manufacturing a cell culture substrate of the present embodiment, the second liquid is a suspension including the second fixative and the cells.

In the method for manufacturing a cell culture substrate of the present embodiment, a recess is formed in the substrate at a position where the film is to be formed, and the film fills the interior of the recess.

In the method for manufacturing a cell culture substrate of the present embodiment, a diameter of the droplet is preferably equal to or less than a film thickness of the film as defined by a depth of the recess.

In the method for manufacturing a cell culture substrate of the present embodiment, at least parts of the first liquid forming the film and the second liquid forming the droplets are mixed to form the particles of hydrogel corresponding to the size of the droplets in the droplet ejecting step.

In the method for manufacturing a cell culture substrate of the present embodiment, the particles encapsulate the cells and are attached to the surface of the substrate.

The method for manufacturing a cell culture substrate of the present embodiment is based on the knowledge that, in the methods for manufacturing a cell culture substrate of the related art, there is a problem in that it is difficult to uniformly coat the first liquid at a desired thickness due to the first liquid being attracted to the wall surface of the well or the like due to surface tension. In addition, the above method is based on the knowledge that, in the methods for manufacturing a cell culture substrate of the related art, since it is difficult to uniformly coat the first liquid at a desired thickness for the reasons described above, there are problems in that the shape of the hydrogel particles may become non-uniform, cells may not be encapsulated in the hydrogel particles and may leak out, the shape of the hydrogel particles may become flat, and the hydrogel particles may not be able to sufficiently contain moisture, causing damage to the cells due to drying.

In order to solve the problems described above, in the method for manufacturing a cell culture substrate of the present embodiment, a recess is formed in the surface of the substrate and the first liquid is added such that the film thickness of the first liquid is equal to the depth of the recess, thereby making it possible to uniformly form a film of the first liquid at the desired thickness and to obtain uniformly shaped hydrogel particles including cells. Due to this, it is possible to allow cells to grow efficiently within the hydrogel particles.

<Cell Culture Substrate>

### FIRST EMBODIMENT

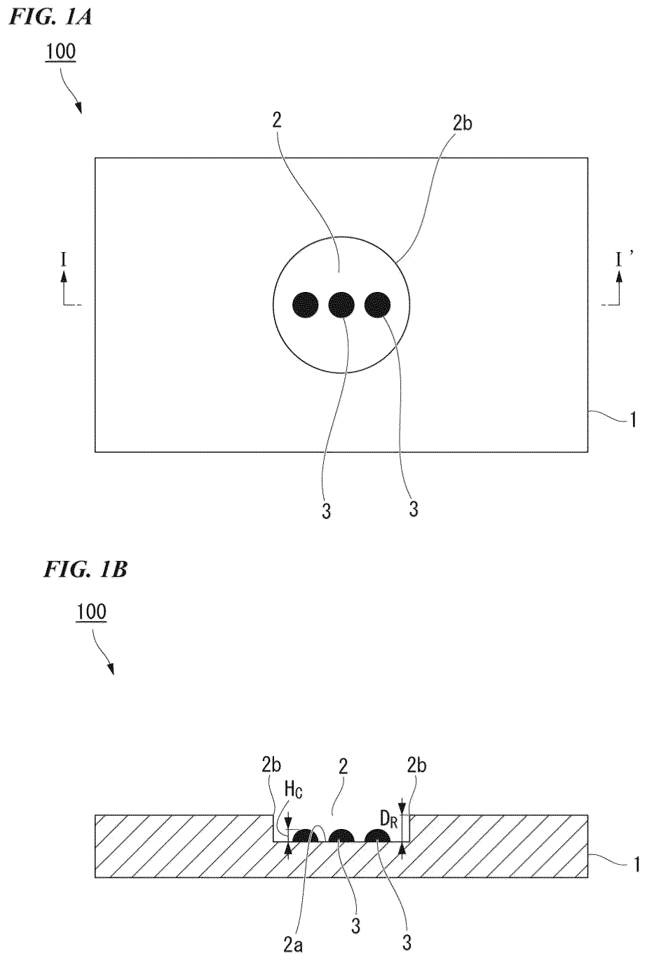

FIG. 1A and FIG. 1B are diagrams showing a cell culture substrate 100 according to the first embodiment of the present invention. FIG. 1A is a plan view of the cell culture substrate 100. FIG. 1B is a cross-sectional view of the cell culture substrate 100 cut along a plane passing through the I-I′ line shown in FIG. 1A.

As shown in FIG. 1A and FIG. 1B, the cell culture substrate 100 has a substrate 1 and hydrogel particles 3 that attach to the surface of the substrate 1 and encapsulate cells. A recess 2 is formed in the surface of the substrate 1. The hydrogel particles 3 are attached to a bottom surface 2a of the recess and a height R of the hydrogel particles is smaller than a depth D_{R} of the recess.

According to the method for manufacturing a cell culture substrate of the present embodiment described below, as shown in FIG. 1A and FIG. 1B, it is possible to obtain the cell culture substrate 100 in which the hydrogel particles 3, that is, the cells, are arranged in the desired position. In addition, as shown in FIG. 1A and FIG. 1B, the hydrogel particles 3 attaching to the bottom surface 2a of the recess makes it possible for the cells to adhere to the substrate 1 via the hydrogel.

(Substrate)

The size, shape, structure, material, and the like of the substrate are not particularly limited as long as cell activity and growth are not inhibited and are able to be selected for the purpose as appropriate.

The size of the substrate is not particularly limited and is able to be selected for the purpose as appropriate.

The shape of the substrate is not particularly limited and is able to be selected for the purpose as appropriate and examples thereof include three-dimensional shapes such as dishes, multi-plates, flasks, or cell inserts; flat shapes such as glass plates, glass slides, and cover glasses; flat film shapes, or the like.

The structure of the substrate is not particularly limited and is able to be selected for the purpose as appropriate and examples thereof include a porous structure, a mesh structure, a concavo-convex structure, a honeycomb structure, or the like.

Examples of the materials of the substrate include organic materials, inorganic materials, and the like. The above may be used alone or may be used in a combination of two or more types.

The organic materials are not particularly limited and are able to be selected for the purpose as appropriate and examples thereof include acrylic materials such as polyethylene terephthalate (PET), polystyrene (PS), polycarbonate (PC), triacetyl cellulose (TAC), polyimide (PI), nylon (Ny), low-density polyethylene (LDPE), medium density polyethylene (HDPE), vinyl chloride, vinylidene chloride, polyphenylene sulfide, polyethersulfone, polyethylene naphthalate, polypropylene, and urethane acrylate, cellulose, and the like.

Inorganic materials are not particularly limited and are able to be selected for the purpose as appropriate and examples thereof include glass, ceramics, and the like.

(Recess)

The recess 2 is formed in the surface of the substrate 1.

Any number of the recesses 2 may be formed in the substrate 1, for example, 1, 2, 3, 4, 5, 6, 7, 8, 9, 10, 12, 24, 48, 96, 384, 1536, or the like.

The depth D_{R} of the recess 2 is preferably 50 μm or more and 500 μm or less. The depth D_{R} of the recess 2 being the lower limit value described above or more makes it possible to obtain hydrogel particles with a more uniform hemispherical or approximately hemispherical shape. On the other hand, by the depth D_{R} of the recess 2 being the upper limit value described above or less, the hydrogel formation speed (curing speed) due to the reaction between the first fixative included in the film of the first liquid and the second fixative included in the droplets of the second liquid becomes more favorable and it is possible for the hydrogel particles to reach the bottom surface 2a of the recess in a half-cured state and thus to adhere the formed hydrogel particles thereto.

The shape of the recess when viewed in plan view is not particularly limited and is able to be selected for the purpose as appropriate and examples thereof include circular shapes, approximately circular shapes, triangular shapes, rectangular shapes, and the like.

For example, in a case where the shape of the recess 2 is a circular shape when viewed in plan view, the inner diameter of the recess 2 is preferably 5 mm or more and 100 mm or less and more preferably 6 mm or more and 20 mm or less.

In the cell culture substrate 100, in a case where the second liquid is added by the ink jet method, the bottom surface 2a of the recess is the landing surface of the droplets of the second liquid, that is, the hydrogel particle adhesion surface.

In the cell culture substrate 100, the recess 2 is formed of the same material as the substrate 1 and it is possible to use any processing method selected for the purpose when forming the recess 2 in the substrate 1. That is, for example, formation is possible by drilling processing using a machining center or the like, optical micromachining processing using a laser or the like, processing by photolithography, etching processing, embossing processing, or the like. In addition, formation is also possible by, for example, injection molding, press molding, stereolithography, and the like.

Among the above, laser processing is preferable as the method for forming the recess 2. When the method for forming the recess 2 is laser processing, it is possible to form the shape of the recess 2 easily and finely. In addition, there is an advantage in that biocompatibility is easy to obtain since there is no processing in direct contact with the substrate 1.

In the cell culture substrate 100, an edge 2b of the recess may have a water repellent property. For example, it is possible to impart a water repellent property by arranging a water repellent layer on the edge 2b of the recess. Alternatively, the edge 2b of the recess may be formed of a material having a water repellent property. The edge 2b of the recess having a water repellent property stops the first liquid added in the film forming step described below spreading on the surface of the substrate 1 and makes it possible to form the film of the first liquid in the recess 2 with a more uniform thickness.

At the edge 2b of the recess, the water contact angle of the surface having a water repellent property is typically 90° or more, preferably 100° or more, and more preferably 105° or more. The water contact angle being the lower limit value described above or more stops the first liquid added in the film forming step described below spreading on the surface of the substrate 1 and makes it possible to form the film of the first liquid in the recess 2 with a more uniform thickness. The water contact angle of the surface having a water repellent property is preferably 150° or less. In the present specification, the water contact angle refers to the water contact angle measured at 23° C.

It is possible to carry out a water repellent treatment by carrying out coating with silane coupling agents having long-chain alkyl groups, with fluorinated silane coupling agents, or the like.

Examples of materials having a water repellent property include fluorine- and oxygen-containing materials, perfluorocarbon materials, and the like and, specifically, ionic fluoropolymers such as Nafion (registered trademark), which is a copolymer of tetrafluoroethylene and perfluoro[2-(fluorosulfonyl ethoxy)propyl vinyl ether].

(Hydrogel Particles)

The hydrogel particles 3 encapsulate cells.

In the hydrogel particles 3, the cells are coated at least partially, preferably entirely, with the hydrogel, and, in addition to the hydrogel, the first fixative and second fixative described below may coexist therewith. In the hydrogel particles 3, the hydrogel coating at least a portion of the cells makes it possible to prevent the cells from drying.

The hydrogel forming the hydrogel particles 3 has a polymer formed by bonding the first fixative and the second fixative, and water included in the polymer. A detailed description will be given of the first fixative, the second fixative, and the polymer to be formed in the method for manufacturing a cell culture substrate described below.

Any number of the hydrogel particles 3 may be arranged on the bottom surface 2a of the recess, for example, 1, 2, 3, 4, 5, 6, 7, 8, 9, 10, 12, 24, 48, 96, 384, 1536, or the like.

The shape of the hydrogel particles is hemispherical or approximately hemispherical.

A height H_{C} of the hydrogel particles may be smaller than the depth D_{R} of the recess described above, for example, more than 10 μm and less than 500 μm and preferably 30 μm or more and 400 μm or less.

The diameter of the hydrogel particles when viewed in plan view is preferably 50 μm or more and 400 μm or less and more preferably 100 μm or more and 200 μm or less.

(Cells)

The cells encapsulated in the hydrogel particles are not particularly limited in terms of type or the like and are able to be selected for the purpose as appropriate and, in terms of taxonomy, use is possible for all cells, regardless of being eukaryotic cells, prokaryotic cells, multicellular organism cells, or unicellular organism cells, for example.

Examples of eukaryotic cells include animal cells, insect cells, plant cells, fungi, and the like. The above may be used alone or may be used in a combination of two or more types. Among the above, animal cells are preferable, and in a case where the cells form cell aggregates, adhesive cells, in which cells adhere to each other and have a degree of cell adhesion that prevents isolation without performing a physicochemical treatment, are more preferable.

Adhesive cells are not particularly limited and are able to be selected for the purpose as appropriate and examples thereof include differentiated cells, undifferentiated cells, and the like.

Examples of differentiated cells include hepatocytes, which are parenchymal cells of the liver; astrocytes; Kupffer cells; vascular endothelial cells; endothelial cells such as sinusoidal endothelial cells and corneal endothelial cells; fibroblasts; osteoblasts; osteoclasts; periodontal ligament-derived cells; epidermal cells such as epidermal keratinocytes; epithelial cells such as tracheal epithelial cells, gastrointestinal epithelial cells, cervical epithelial cells, and corneal epithelial cells; mammary gland cells; pericytes; muscle cells such as smooth muscle cells and cardiomyocytes; renal cells; pancreatic islet of Langerhans cells; nerve cells such as peripheral nerve cells and optic nerve cells; chondrocytes, and the like. The adhesive cells may be primary cells taken directly from tissues or organs, or may be several generations from the above.

The undifferentiated cells are not particularly limited and are able to be selected for the purpose as appropriate and examples thereof include pluripotent stem cells such as embryonic stem cells that are undifferentiated cells or mesenchymal stem cells having pluripotency; unipotent stem cells such as vascular endothelial progenitor cells having unipotency; iPS cells, and the like.

In addition to the recess 2 of the cell culture substrate according to the first embodiment, the cell culture substrate of the present embodiment is further provided with a well 4, as shown in the cell culture substrates according to the second, fourth, and sixth embodiments described below. By providing the well 4, it is possible to stably culture the cells in the hydrogel particles for a longer period of time. In addition, in the cell culture substrate of the present embodiment, the recess 2 of the cell culture substrate according to the first embodiment may also be formed by arranging a convex strip 5, as shown in the cell culture substrates according to the three to sixth embodiments.

Next, a detailed description will be given below of the cell culture substrate according to each embodiment.

### SECOND EMBODIMENT

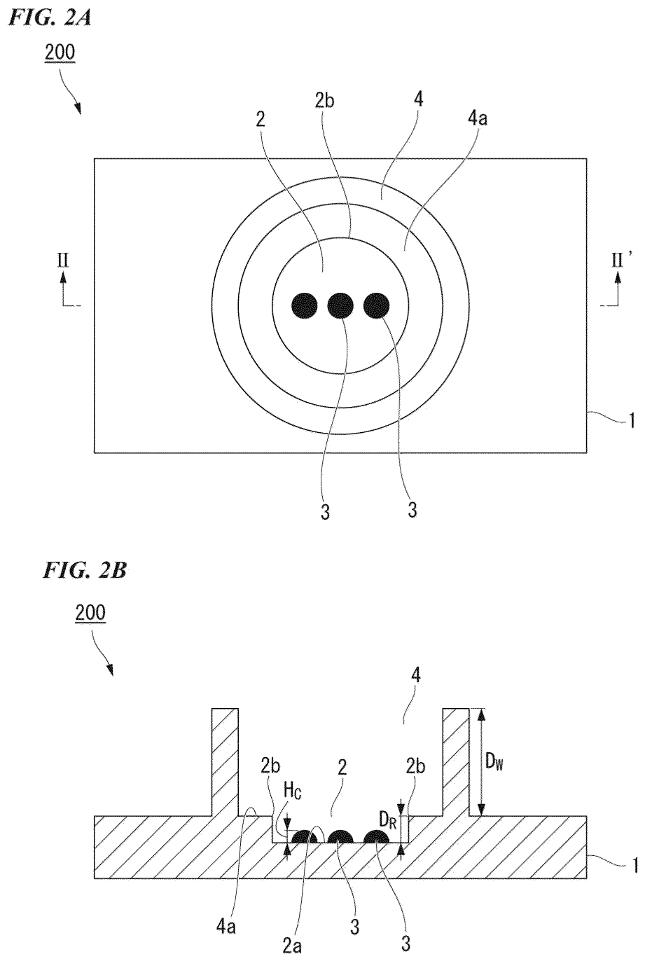

FIG. 2A and FIG. 2B are diagrams showing a cell culture substrate 200 according to the second embodiment of the present invention. FIG. 2A is a plan view of the cell culture substrate 200. FIG. 2B is a cross-sectional view of the cell culture substrate 200 cut along a plane passing through the II-II′ line shown in FIG. 2A. The cell culture substrate 200 shown in FIG. 2A and FIG. 2B differs from the cell culture substrate 100 shown in FIG. 1A and FIG. 1B in the point of being further provided with the well 4. Providing the well 4 in the cell culture substrate 200 makes it possible to increase the storable solution volume to be larger than in the case of the recess 2 alone and reduces the influence of drying. Therefore, it is possible to stably culture the cells in the hydrogel particles for a longer period of time.

In the cell culture substrate 200 shown in FIG. 2A, the substrate is a well plate and the recess 2 is formed at the bottom of one or more of the wells 4 of the well plate.

(Wells)

Any number of the wells 4 may be formed in the substrate 1, for example, 1, 2, 4, 6, 12, 24, 48, 96, 384, 1536, or the like.

The shape of the well 4 when viewed in plan view is not particularly limited and is able to be selected for the purpose as appropriate and examples thereof include circular shapes, approximately circular shapes, triangular shapes, rectangular shapes, and the like.

For example, in a case where the shape of the well 4 is a circular shape when viewed in plan view, the inner diameter of the well 4 is preferably 5 mm or more and 300 mm or less and more preferably 6 mm or more and 20 mm or less.

A depth D_{W} of the well 4 may be greater than the depth D_{R} of the recess 2, for example, 1 mm or more and 20 mm or less is possible.

The arrangement of the plurality of the wells 4 on the substrate 1 is not particularly limited and is able to be selected for the purpose as appropriate, for example, the plurality of the wells 4 may be arranged to be aligned at equal intervals horizontally and vertically.

In the plurality of the wells 4, a recess may be formed at the bottom of one or more of the wells 4 and recesses are preferably formed at the bottoms of all of the wells 4.

It is possible to set the volume of the wells 4 as appropriate according to the type of cells and the size of the hydrogel particles, for example, 100 μL or more and 1000 μL or less is possible. Setting the volume of the well 4 to the lower limit value described above or more makes it possible to increase the storable solution volume to be larger than in the case of the recess 2 alone and reduces the influence of drying. In addition, it is possible to easily control and manage the liquid level and to stably culture the cells.

In the cell culture substrate 200, the recess 2 and the well 4 are formed of the same material as the substrate 1 and it is possible to form the recess 2 and the well 4 by the same known processing methods as the method for forming the recess 2 described in the cell culture substrate 100 according to the first embodiment above.

In the cell culture substrate 200, the edge 2b of the recess or the bottom surface 4a of the well, which is the outer edge region of the recess 2 when viewed in plan view as shown in FIG. 2A, may have a water repellent property. For example, it is possible to impart a water repellent property by arranging a water repellent layer on the edge 2b of the recess and the bottom surface 4a of the well. Alternatively, the edge 2b of the recess and the bottom surface 4a of the well may be formed of a material having a water repellent property. The edge 2b of the recess or the bottom surface 4a of the well having a water repellent property stops the first liquid added in the film forming step described below spreading on the surface of the substrate 1 and makes it possible to form the film of the first liquid in the recess 2 with a more uniform thickness.

The water contact angle of the surface having a water repellent property, the water repellent treatment method, and the material having a water repellent property are as described in the cell culture substrate 100 according to the first embodiment above.

### THIRD EMBODIMENT

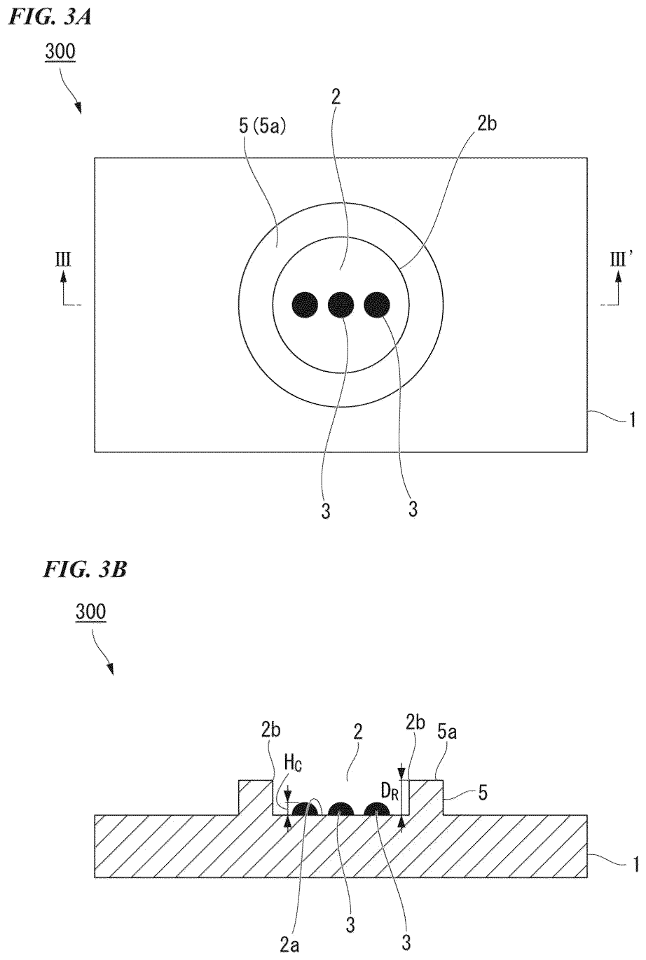

FIG. 3A and FIG. 3B are diagrams showing a cell culture substrate 300 according to the third embodiment of the present invention. FIG. 3A is a plan view of the cell culture substrate 300. FIG. 3B is a cross-sectional view of the cell culture substrate 300 cut along a plane passing through the line shown in FIG. 3A. The cell culture substrate 300 shown in FIG. 3A and FIG. 3B differs from the cell culture substrate 100 shown in FIG. 1A and FIG. 1B in the point that the recess 2 is a portion surrounded by the convex strip 5 provided in a ring shape in the substrate 1.

(Convex Strip)

The convex strip 5 is formed on the surface of the substrate 1.

The shape and material of the convex strip 5 are not particularly limited as long as it is possible to uniformly maintain the film thickness of the first liquid.

Any number of the convex strips 5 may be formed on the substrate 1, for example, 1, 2, 3, 4, 5, 6, 7, 8, 9, 10, 12, 24, 48, 96, 384, 1536, or the like.

The height of the convex strip 5, that is, the depth D_{R} of the recess 2, is preferably 50 μm or more and 500 μm or less. The depth D_{R} of the recess 2 being the lower limit value described above or more makes it possible to obtain hydrogel particles with a more uniform hemispherical or approximately hemispherical shape. On the other hand, by the depth D_{R} of the recess 2 being the upper limit value described above or less, the hydrogel formation speed (curing speed) due to the reaction between the first fixative included in the film of the first liquid and the second fixative included in the droplets of the second liquid becomes more favorable and it is possible for the hydrogel particles to reach the bottom surface 2a of the recess in a half-cured state and thus it is possible to adhere the formed hydrogel particles thereto.

The shape of the convex strip 5 when viewed in plan view may be ring-shaped, such as a circular ring shape or an approximately circular ring shape; or a polygonal ring shape such as a triangle shape or a rectangular shape.

For example, in a case where the shape of the convex strip 5 is a circular ring shape in plan view, the width of the convex strip 5 is not particularly limited and may be, for example, 0.5 mm, 0.6 mm, 0.7 mm, 0.8 mm, 0.9 mm, 1 mm, or the like. It is possible to set the width of the convex strip 5 as appropriate according to the location where the convex strip is to be provided, the size of the recess 2 to be secured, and the like.

In addition, in a case where the shape of the convex strip 5 is a circular ring shape in plan view, the inner diameter of the convex strip 5, that is, the inner diameter of the recess 2, is preferably 5 mm or more and 100 mm or less and more preferably 6 mm or more and 20 mm or less.

In the cell culture substrate 300, the recess 2 and the convex strip 5 are formed of the same material as the substrate 1 and it is possible to form the recess 2 and the convex strip 5 by the same known processing method as the method for forming the recess 2 described in the cell culture substrate 100 according to the first embodiment above.

In the cell culture substrate 300, the edge 2b of the recess or an upper surface 5a of the convex strip 5 may have a water repellent property. For example, it is possible to impart a water repellent property by arranging a water repellent layer on the edge 2b of the recess and the upper surface 5a of the convex strip 5. Alternatively, the edge 2b of the recess and the upper surface 5a of the convex strip 5 may be formed of a material having a water repellent property. The edge 2b of the recess or the convex strip 5 having a water repellent property stops the first liquid added in the film forming step described below spreading on the surface of the substrate 1 and makes it possible to form the film of the first liquid in the recess 2 with a more uniform thickness.

The water contact angle of the water repellent surface, the water repellent treatment method, and the material having a water repellent property are as described in the cell culture substrate 100 according to the first embodiment above.

### FOURTH EMBODIMENT

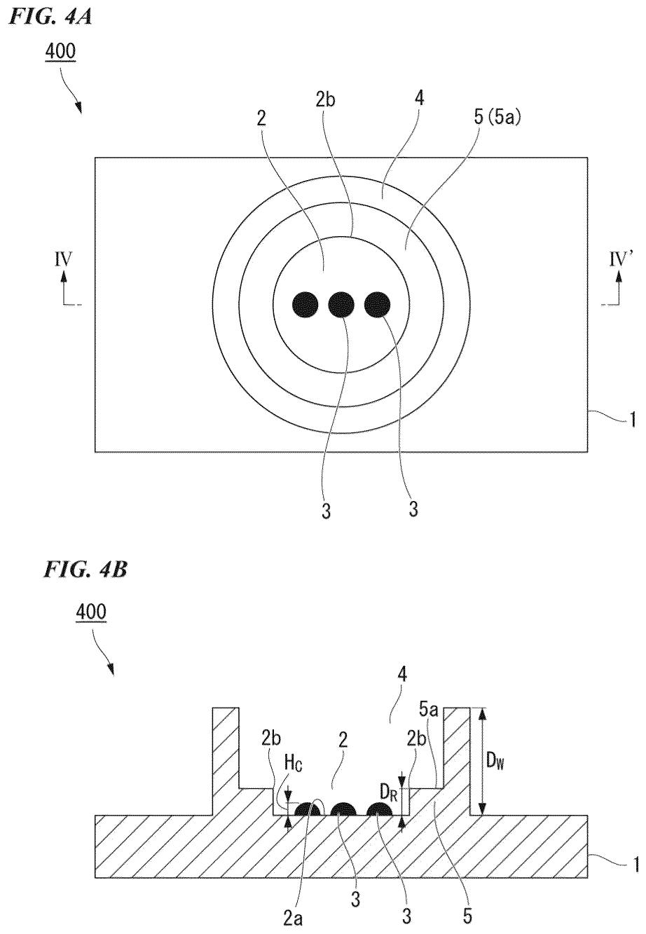

FIG. 4A and FIG. 4B are diagrams showing a cell culture substrate 400 according to the fourth embodiment of the present invention. FIG. 4A is a plan view of the cell culture substrate 400. FIG. 4B is a cross-sectional view of the cell culture substrate 400 cut along a plane passing through the IV-IV′ line shown in FIG. 4A. The cell culture substrate 400 shown in FIG. 4A and FIG. 4B differs from the cell culture substrate 300 shown in FIG. 3A and FIG. 3B in the point of being further provided with the well 4. Providing the well 4 in the cell culture substrate 400 makes it possible to increase the storable solution volume to be larger than in the case of the recess 2 alone and reduces the influence of drying. Therefore, it is possible to stably culture cells in the hydrogel particles for a longer period of time.

In the cell culture substrate 400, the recess 2, the well 4, and the convex strip 5 are formed of the same material as the substrate 1 and it is possible to form the recess 2, the well 4, and the convex strip 5 by the same known processing method as the method for forming the recess 2 described in the cell culture substrate 100 according to the first embodiment above.

In the cell culture substrate 400, the edge 2b of the recess or the upper surface 5a of the convex strip 5 may have a water repellent property. For example, it is possible to impart a water repellent property by arranging a water repellent layer on the edge 2b of the recess and the upper surface 5a of the convex strip 5. Alternatively, the edge 2b of the recess and the upper surface 5a of the convex strip 5 may be formed of a material having a water repellent property. The edge 2b of the recess or the convex strip 5 having a water repellent property stops the first liquid added in the film forming step described below spreading on the surface of the substrate 1 and makes it possible to form the film of the first liquid in the recess 2 with a more uniform thickness.

The water contact angle of the water repellent surface, the water repellent treatment method, and the material having a water repellent property are as described in the cell culture substrate 100 according to the first embodiment above.

### FIFTH EMBODIMENT

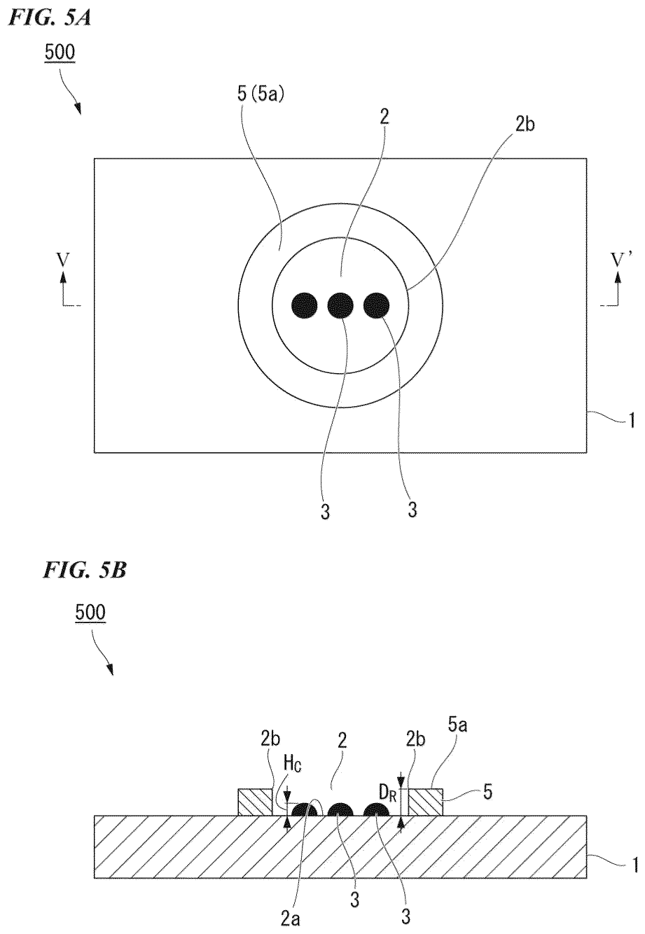

FIG. 5A and FIG. 5B are diagrams showing a cell culture substrate 500 according to the fifth embodiment of the present invention. FIG. 5A is a plan view of the cell culture substrate 500. FIG. 5B is a cross-sectional view of the cell culture substrate 500 cut along a plane passing through the V-V line shown in FIG. 5A. The cell culture substrate 500 shown in FIG. 5A and FIG. 5B differs from the cell culture substrate 300 shown in FIG. 3A and FIG. 3B in the point that the convex strip 5 is formed of a separate member from the substrate 1. In the cell culture substrate 300, the convex strip 5 may be fixed to the substrate 1 or may be formed of a member removable from the substrate and is preferably formed of a member removable from the substrate. By the convex strip 5 being formed of a member removable from the substrate, for example, in a case where the substrate is a glass slide, it is possible to remove the convex strip 5 before observation so as to not interfere with the observation of the cells in the hydrogel particles.

(Convex Strip)

In a case where the convex strip 5 is formed of a separate member from the substrate 1, examples of the shapes of the convex strip 5 are the same as exemplified in the third embodiment described above.

Examples of the material of the convex strip 5 include silicone rubber, metal, glass, plastic, rubber, and the like. The convex strip 5 may be formed of a material having a water repellent property. Examples of the material having a water repellent property are the same as exemplified in the cell culture substrate 100 according to the first embodiment described above. In particular, in a case where the convex strip 5 is a removable member, a member in which the material is silicone rubber is preferable due to being easy to process and exhibiting almost no toxicity to cells.

In the cell culture substrate 500, it is possible to form the convex strip 5 by arranging a convex strip produced by processing a sheet formed of the material described above into a desired shape by a known processing method such as punching processing or laser processing, on the substrate 1.

In the cell culture substrate 500, the edge 2b of the recess or the upper surface 5a of the convex strip 5 may have a water repellent property. For example, it is possible to impart a water repellent property by arranging a water repellent layer on the edge 2b of the recess and the upper surface 5a of the convex strip 5. Alternatively, the edge 2b of the recess and the upper surface 5a of the convex strip 5 may be formed of a material having a water repellent property. The edge 2b of the recess or the convex strip 5 having a water repellent property stops the first liquid added in the film forming step described below spreading on the surface of the substrate 1 and makes it possible to form the film of the first liquid in the recess 2 with a more uniform thickness.

### SIXTH EMBODIMENT

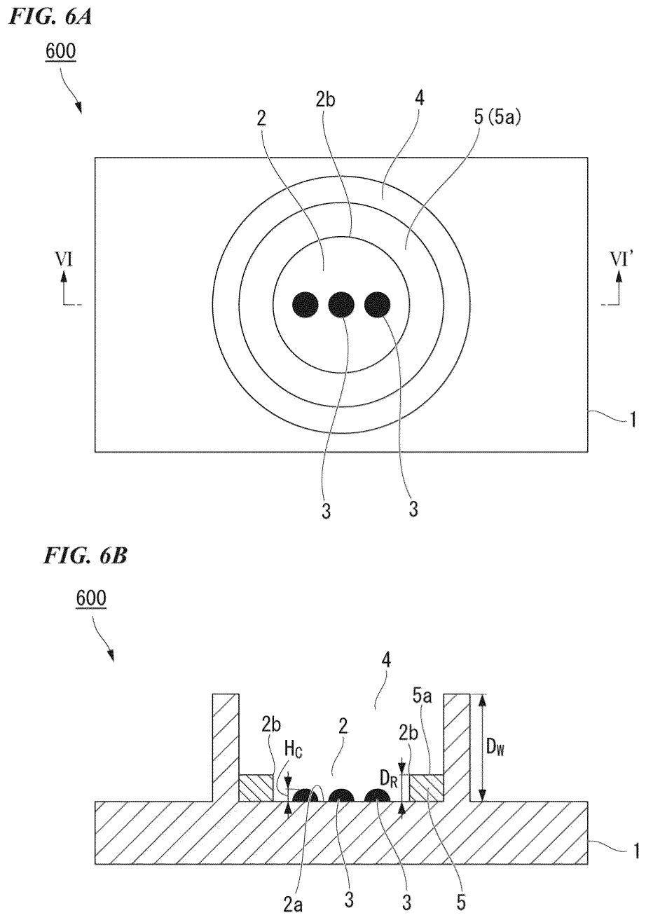

FIG. 6A and FIG. 6B are diagrams showing a cell culture substrate 600 according to the sixth embodiment of the present invention. FIG. 6A is a plan view of the cell culture substrate 600. FIG. 6B is a cross-sectional view of the cell culture substrate 600 cut along a plane passing through the VI-VI′ line shown in FIG. 6A. The cell culture substrate 600 shown in FIG. 6A and FIG. 6B differs from the cell culture substrate 500 shown in FIG. 5A and FIG. 5B in the point of being further provided with the well 4. Providing the well 4 in the cell culture substrate 600 makes it possible to increase the storable solution volume to be larger than in the case of the recess 2 alone and reduces the influence of drying. Therefore, it is possible to stably culture the cells in the hydrogel particles for a longer period of time.

In the cell culture substrate 600, the edge 2b of the recess or the upper surface 5a of the convex strip 5 may have a water repellent property. For example, it is possible to impart a water repellent property by arranging a water repellent layer on the edge 2b of the recess and the upper surface 5a of the convex strip 5. Alternatively, the edge 2b of the recess and the upper surface 5a of the convex strip 5 may be formed of a material having a water repellent property. The edge 2b of the recess or the convex strip 5 having a water repellent property stops the first liquid added in the film forming step described below spreading on the surface of the substrate 1 and makes it possible to form the film of the first liquid in the recess 2 with a more uniform thickness.

The embodiments described above may each be implemented alone or may be implemented in a combination of two or more types thereof.

The cell culture substrate of the present embodiment is not limited to the cell culture substrates shown in FIG. 1A to FIG. 6B, but may be a cell culture substrate shown in FIG. 1A to FIG. 6B in which a part of the configuration is changed or removed, or a cell culture substrate to which other configurations are added to the configurations described so far, in a range in which the effects of the present invention are not impaired.

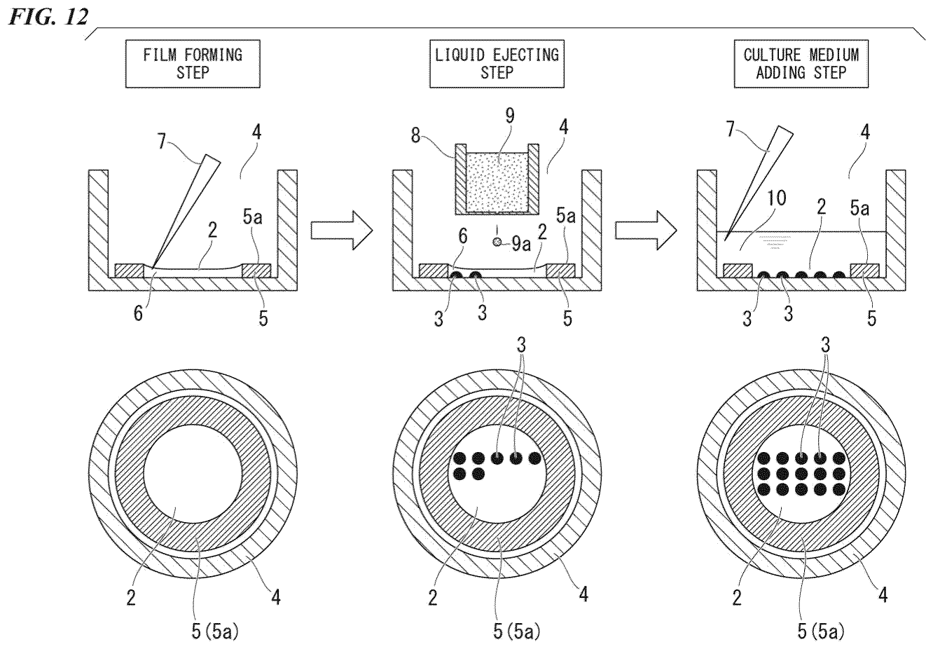

For example, in the cell culture substrate 600 shown in FIG. 6A and FIG. 6B, the convex strip 5 is arranged to be in contact with the wall surface of the well 4; however, as shown in FIG. 12, the convex strip 5 may be arranged at a certain distance from the wall surface of the well 4. Such an arrangement makes it easier to attach and detach the convex strip 5 in a case where the convex strip 5 is formed of a removable member.

For example, in the cell culture substrates shown in FIG. 1A to FIG. 6B, the recess 2 or the interior of the well 4 may be filled with a culture medium. Due to this, drying of the hydrogel particles is prevented and it is possible for the cells to be stably cultured for a longer period of time.

For example, in the cell culture substrates shown in FIG. 1A and FIG. 1B, FIG. 3A and FIG. 3B, and FIG. 5A and FIG. 5B, it is also possible to culture cells fixed via the hydrogel by immersing the cell culture substrates in a separate container to which a culture medium or the like is added.

It is possible to preferably use the cell culture substrate of the present embodiment as a substrate for evaluating the toxicity or drug efficacy of a test substance in vitro.

Next, a detailed description will be given below of each step of the method for manufacturing a cell culture substrate of the present embodiment.

<Film Forming Step>

In the film forming step, a film of the first liquid is formed on the surface of the substrate. Recesses are formed in the substrate at the position where the film is to be formed and the film fills the interior of the recesses. That is, in the film forming step, the film thickness of the first liquid is defined by the depth of the recess and the first liquid is added such that the film thickness of the first liquid is equal to the depth of the recess.

In the related art, the film thickness is not stable because the first liquid is attracted to the wall surface of the well or the like that is the dissolution chamber that stores the first liquid. In contrast, in the method for manufacturing a cell culture substrate of the present embodiment, the first liquid is added such that the film thickness of the first liquid is equal to the depth of the recess 2, whereby it is possible to make the film thickness of the first liquid uniform and to form uniformly shaped hydrogel particles in the droplet ejecting step described below. Here, “the film thickness of the first liquid is equal to the depth of the recess 2” means that the film thickness of the first liquid is 80% or more and 100% or less of the depth of the recess 2. It is possible to adjust the film thickness of the first liquid as appropriate according to the depth of the recess 2 and, specifically, it is possible to set the film thickness to, for example, 40 μm or more and 500 μm or less.

In the film forming step, the method for adding the first liquid to the interior of the recess 2 is not particularly limited and is able to be selected for the purpose as appropriate and examples thereof include a pipette dispensing method using a micropipette or the like, a micromanipulator method, an aspirator method, a droplet ejecting method (ink jet method), a gel extrusion method, transfer methods such as a screen-printing method, and the like.

[First Liquid]

The first liquid is an aqueous solution including the first fixative.

(First Fixative)

The first fixative is not particularly limited as long as the first fixative is mixed with the second fixative to form a cross-linked structure and is able to be selected for the purpose as appropriate. Among the above, fixatives that improve the thickening property of the hydrogel to be formed are preferable. Specific examples of the first fixative include biologically derived polymers such as collagen, elastin, gelatin, and fibroin; coagulation factors such as fibrinogen; adhesion factors such as fibronectin, laminin, and recombinant peptide; polysaccharide compound metal salts such as alginic acid and gellan gum; synthetic polymers such as polylactic acid and polyethylene glycol, and the like. In addition, it is also possible to use the polymers described above modified with functional groups such as thiol groups. The above may be used alone or may be used in a combination of two or more types. Among the above, thiol gelatin is preferable.

(Other Components)

The first liquid may further include other components as necessary.

The other components are not particularly limited and are able to be selected for the purpose as appropriate and examples thereof include culture media, cross-linking agents, pH adjusters, preservatives, antioxidants, and the like.

It is possible to prepare the first liquid by adding the first fixative and, as necessary, the other components to a buffer solution such as water or phosphate buffered saline to a desired concentration, and carrying out mixing.

<Droplet Ejecting Step>

In the droplet ejecting step, droplets 9a of the second liquid are ejected onto the film 6. In the droplet ejecting step, at least parts of the first liquid forming the film and the second liquid forming the droplets 9a are mixed to form particles 3 of hydrogel corresponding to the size of the droplets 9a. The particles 3 encapsulate cells and are attached to the surface of the substrate 1.

In the droplet ejecting step, the diameter of the droplets 9a is equal to or less than the film thickness of the film defined by the depth of the recess. Due to this, it is possible to obtain uniformly shaped hydrogel particles by curing the hydrogel to an appropriate hardness by the time of reaching the bottom surface of the recess.

Specifically, it is possible for the diameter of the droplets 9a to be more than 20 μm and 500 μm or less, and 50 μm or more and 400 μm or less is preferable.

In the droplet ejecting step, it is possible to select the method for ejecting the droplets 9a for the purpose as appropriate as long as the method is able to impart the droplets 9a to the desired position and examples thereof include an ink jet type ejecting method, a micromanipulator method, a gel extrusion method, transfer methods such as a screen-printing method, or the like. Among the above, the ink jet type ejecting method is preferable. The ink jet type ejecting method (may be referred to below as “ink jet method”) is a method in which droplets are continuously dropped from a nozzle.

Examples of ink jet methods include on-demand methods, continuous methods, and the like. Among the above, in the case of the continuous method, the dead volume of the suspension to be used tends to increase for reasons such as continuing to perform empty ejecting until a stable ejection state is reached, adjustment of droplet volume, and continuous droplet formation even when moving between each well of the well plate. In the present embodiment, it is preferable to reduce the influence of dead volume from the viewpoint of adjusting the number of cells and for this reason the on-demand method is the more suitable of the two methods described above.

Examples of the on-demand method include a plurality of known methods such as the pressure-applied method, which ejects liquid by applying pressure to the liquid, the thermal method, which ejects liquid by heating and boiling a film, the electrostatic method, which forms droplets by pulling droplets using electrostatic attraction, and the like. Among the above, the pressure-applied method is preferable for the following reasons.

In the electrostatic method, it is necessary for an electrode to be placed opposite the ejecting portion that holds the suspension and forms droplets. In the method for manufacturing a substrate for evaluation of the present invention, the substrate for receiving the droplets is arranged opposite thereto and it is preferable not to arrange an electrode there in order to increase the degree of freedom in the substrate configuration.

In the thermal method, since local heating is generated, there are concerns regarding the influence on cells, which are biomaterials, and scorching (kogation) of the heater portion. Since the influence of heat depends on the content and the application of the substrate, it is not necessary to exclude the thermal method altogether; however, the pressure-applied method is preferable over the thermal method due to the point that there is no concern regarding scorching of the heater portion.

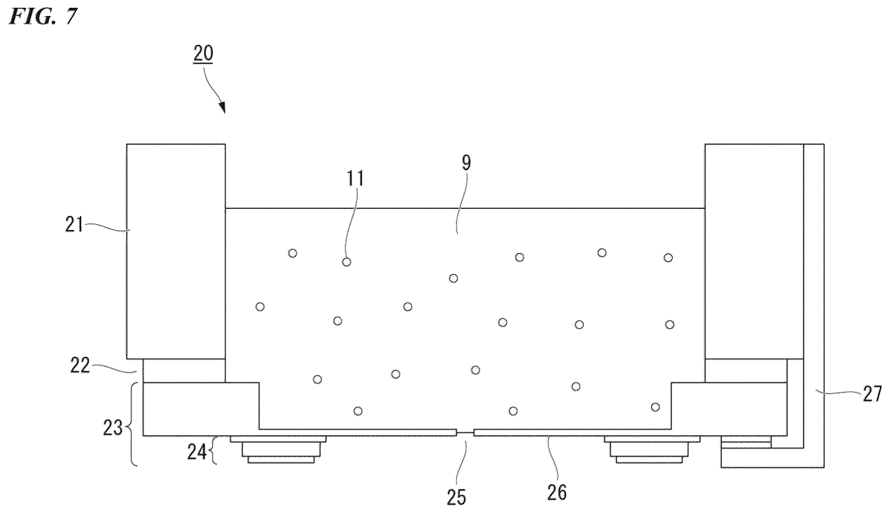

Examples of the pressure-applied method include a method of applying pressure to the liquid using a piezo element, a method of applying pressure to the liquid using a valve such as an electromagnetic valve, and the like. FIG. 7 shows a configuration example of an ejecting head able to be used for ejecting droplets of the second liquid. FIG. 7 is a schematic diagram showing an example of a piezoelectric ejecting head.

In a piezoelectric ejecting head 20, a MEMS chip 23 is arranged on the bottom surface of a liquid chamber member 21 via an elastic member 22.

A membrane 26, which is a thin plate portion, is arranged on the MEMS chip 23. A nozzle 25 is formed in the center of the membrane 26. Thin-film shaped piezoelectric elements 24 are arranged on the outer side surface of the membrane 26. By applying a driving waveform to the piezoelectric elements 24 arranged on the MEMS chip 23 from a driving waveform generation supply source (not shown) through a wiring 27, the membrane 26 of the MEMS chip 23 vibrates and a second liquid 9 in the liquid chamber is ejected from the nozzle 25 as the droplets 9a.

The liquid chamber formed by the liquid chamber member 21 and the membrane 26 in which the nozzle 25 is formed is a liquid holding portion that holds the second liquid 9, which is a suspension including the cells 11. The nozzle 25, which is a through hole, is formed on the lower surface side in the liquid chamber. It is possible to form the liquid chamber, for example, from metal, silicon, ceramics, or the like.

The membrane 26 is a film-shaped member fixed to the lower end portion of the liquid chamber member 21. The nozzle 25, which is a through hole, is formed in the approximate center of the membrane 26 and the second liquid 9 held in the liquid chamber is ejected as droplets from the nozzle 25 by vibration of the membrane 26. Since droplets are formed by the inertia of the vibration of the membrane 26, it is possible to eject even the second liquid 9 having a high surface tension (high viscosity). It is possible for the planar shape of the membrane 26 to be a circular shape, for example, but the shape may also be elliptical, a rectangular shape, or the like.

The material of the membrane 26 is not particularly limited, but it is preferable to use a material with a certain degree of hardness, because when the material is excessively soft, the membrane 26 will vibrate easily and it will be difficult to immediately suppress the vibration when not carrying out the ejection. For example, it is possible to use a metallic material, a ceramic material, a polymer material with a certain degree of hardness, or the like as the material of the membrane 26.

In particular, when using cells, a material that has a low attachment property with respect to cells and proteins is preferable. The cell attachment property is generally said to be dependent on the contact angle of the material with water, and when the material is highly hydrophilic or hydrophobic, the cell attachment property is low. It is possible to use various metallic materials and ceramics (metal oxides) as highly hydrophilic materials and to use fluorine resins or the like as highly hydrophobic materials.

Other examples of such materials include stainless steel, nickel, aluminum, and the like or silicon dioxide, alumina, zirconia, and the like. In addition to the above, it is considered that coating the surface of the material decreases cell adhesion. For example, it is possible to coat the material surface with the metal or metal oxide material described above or to carry out the coating with a synthetic phospholipid polymer that mimics a cell membrane (for example, Lipidure, manufactured by NOF Corporation).

The nozzle 25 is preferably formed as a substantially circular through hole in the approximate center of the membrane 26. In such a case, the diameter of the nozzle 25 is not particularly limited, but is preferably two or more times the size of the cells 11 to avoid the cells 11 clogging the nozzle 25. In a case where the cells 11 are, for example, animal cells, in particular, human cells, the diameter of the nozzle 25 is preferably 10 μm or more and more preferably 100 μm or more, in accordance with the cells to be used, since the size of human cells is generally approximately 5 μm or more and 50 μm or less.

On the other hand, the diameter of the nozzle 25 is preferably 200 μm or less. In other words, in the ejecting head 20, the diameter of nozzle 25 is typically in a range of 10 μm or more and 200 μm or less.

The piezoelectric elements 24 are formed on the lower surface side of the membrane 26. It is possible to design the shape of the piezoelectric elements 24 in accordance with the shape of the membrane 26. For example, in a case where the planar shape of the membrane 26 is a circular shape, the piezoelectric elements 24 are preferably formed with a circular ring shape (ring shape) planar shape at the periphery of the nozzle 25.

By supplying a driving waveform to the piezoelectric elements 24 from a driving waveform generation supply source, it is possible to cause the membrane 26 to vibrate. Due to the vibration of the membrane 26, it is possible to eject droplets containing the cells 11 from the nozzle 25.

It is possible to set the piezoelectric elements 24 to have a structure provided with electrodes for applying a voltage to the upper surface and lower surface of the piezoelectric material. In this case, by applying a voltage between upper and lower electrodes of the piezoelectric elements 24 from a driving waveform generation supply source, it is possible to apply a compressive stress in the transverse direction on the paper surface to vibrate the membrane 26 in the vertical direction on the paper surface. For example, it is possible to use lead zirconate titanate (PZT) as the piezoelectric material. It is also possible to use various other piezoelectric materials, such as bismuth iron oxide, niobate metal oxide, barium titanate, or the above materials with added metals or different oxides.

It is possible for the driving waveform generation supply source to selectively (for example, alternately) impart, to the piezoelectric elements 24, an ejection waveform that vibrates the membrane 26 to form droplets and an agitation waveform that vibrates the membrane 26 in a range in which no droplets are formed.

For example, by making both the ejection waveform and the agitation waveform square waves and lowering the driving voltage of the agitation waveform compared to the driving voltage of the ejection waveform, it is possible to stop droplets from being formed by the application of the agitation waveform. In other words, it is possible to control the vibration state (degree of vibration) of the membrane 26 using the high and low driving voltages.

In the ejecting head 20, since the piezoelectric elements 24 are formed on the lower surface side of the membrane 26, when the membrane 26 is vibrated by the piezoelectric elements 24, it is possible to generate a flow in a direction from the bottom to the top of the liquid chamber.

At this time, the movement of the cells 11 is a motion from the bottom to the top and convection currents are generated in the liquid chamber, causing agitation of the second liquid 9 containing the second fixative and the cells 11. The flow in the direction from the bottom to the top of the liquid chamber uniformly disperses the settled and aggregated cells 11 inside the liquid chamber.

In other words, by the driving waveform generation supply source applying an ejection waveform to the piezoelectric elements 24 and controlling the vibration state of the membrane 26, it is possible to eject the second liquid 9 containing the second fixative and the cells 11 held in the liquid chamber as droplets from the nozzle 25. In addition, by the driving waveform generation supply source applying an agitation waveform to the piezoelectric elements 24 and controlling the vibration state of the membrane 26, it is possible to agitate the second liquid 9 containing the second fixative and the cells 11 held in the liquid chamber. During agitation, droplets are not ejected from the nozzle 25.

In this manner, by agitating the second liquid 9 containing the second fixative and the cells 11 while no droplets are formed, the cells 11 are prevented from settling or aggregating on the membrane 26 and it is possible to evenly disperse the cells 11 in the second liquid 9 containing the second fixative. Due to this, it is possible to suppress clogging of the nozzle 25. As a result, it is possible for the second liquid 9 containing the second fixative and the cells 11 to be ejected continuously and stably as droplets for a long time.

In addition, air bubbles may be mixed in the second liquid 9 containing the second fixative and the cells 11. Even in such a case, since the top of the liquid chamber in the ejecting head 20 is open, it is possible to discharge air bubbles mixed in the second liquid 9 containing the second fixative and the cells 11 to the outside air through the top of the liquid chamber. Due to this, it is possible to form droplets continuously and stably without having to discard a large amount of liquid due to the air bubble discharge.

That is, in a case where air bubbles are mixed in the vicinity of the nozzle 25 or in a case where a large number of air bubbles are mixed in on the membrane 26, the ejection state is influenced, thus, it is necessary to discharge the mixed-in air bubbles in order to form the droplets stably for a long time. Ordinarily, air bubbles mixed in on the membrane 26 move upward naturally or due to the vibration of the membrane 26; however, the top of the liquid chamber is open and thus it is possible to discharge the mixed-in air bubbles from the top of the liquid chamber. Therefore, even when air bubbles are mixed into the liquid chamber, it is possible to prevent the generation of ejection failure and to form droplets continuously and stably.

The membrane 26 may be vibrated at a timing when no droplets are formed and in a range in which no droplets are formed to actively move air bubbles upward in the liquid chamber.

It is possible to use either a piezoelectric or an electromagnetic valve ejecting head, but the pressure-applied method using the electromagnetic valve is not able to form droplets repeatedly at high speed and thus the piezoelectric method is preferably used to increase the manufacturing throughput of the cell culture substrate. As a piezoelectric ejecting head, it is possible to suitably use a single cell printer made by Cytena or the like.

In addition, in a piezoelectric ejecting head using a general piezoelectric element, there may be problems in that uneven cell concentrations may be generated due to sedimentation and nozzle clogging may occur. For this reason, it is possible to preferably use the piezoelectric ejecting head shown in FIG. 7. In the ejecting head 20 in FIG. 7, by applying a driving waveform with respect to the piezoelectric elements 24 from a driving waveform generation supply source (not shown) through the wiring 27, it is possible to apply compressive stress in the transverse direction on the paper surface to deform the membrane 26 in the vertical direction on the paper surface. This makes it possible to solve the above problem.

In the continuous method, periodic fluctuations are applied by piezoelectric elements or heaters when droplets are pressed and extruded from the nozzle, thereby making it possible to continuously put out minute droplets. Furthermore, by carrying out the control by applying a voltage to the droplets in the ejecting direction in flight, it is also possible to choose whether the droplets are to land in the wells or are to be recovered in a recovery portion. Such a method is used in cell sorters or flow cytometers and, for example, it is possible to use the apparatus named “Cell Sorter SH800” manufactured by Sony Corporation.

In the ejecting head, it is possible to form droplets and agitate the second liquid according to the strength or weakness of the voltage. By inputting a plurality of pulses that are not strong enough to eject droplets during the period when droplets are not being ejected, it is possible to agitate the suspension in the liquid chamber and to suppress the generation of concentration distribution due to cell sedimentation.

For details of the voltage applied to the piezoelectric element and the droplet formation operation in the ejecting head, for example, the contents described in Patent Document 1 are preferably also applied to the method for manufacturing a cell culture substrate of the present embodiment.

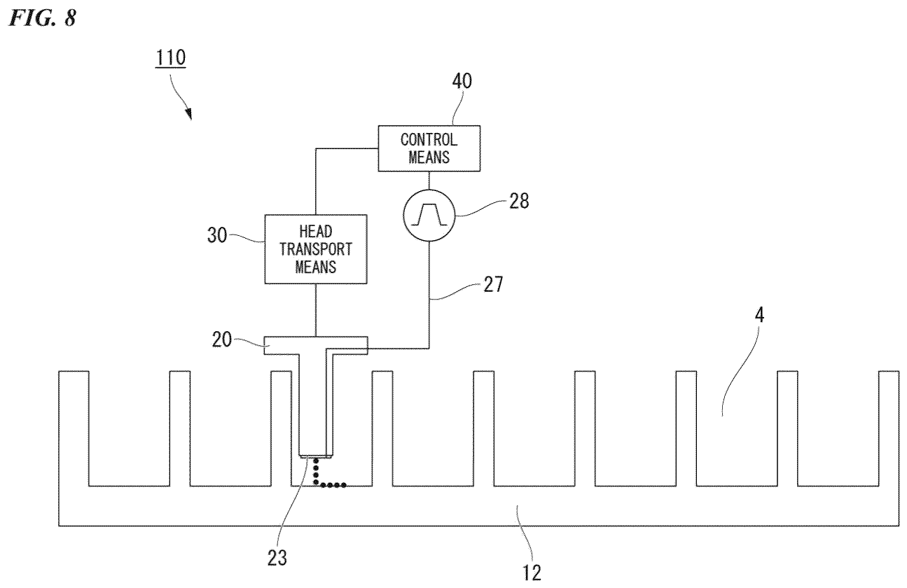

A description will be given of the apparatus used in the method for manufacturing a cell culture substrate of the present embodiment using FIG. 8 to FIG. 10.

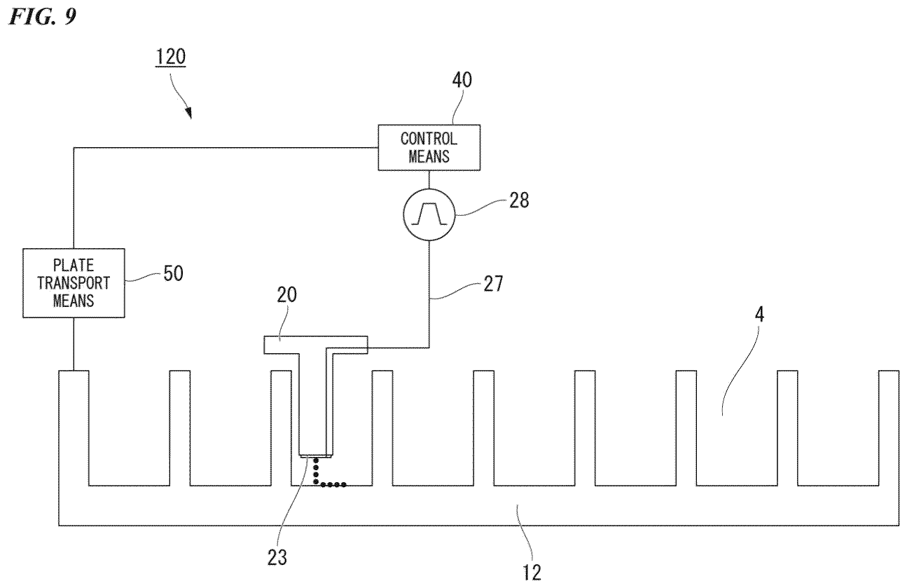

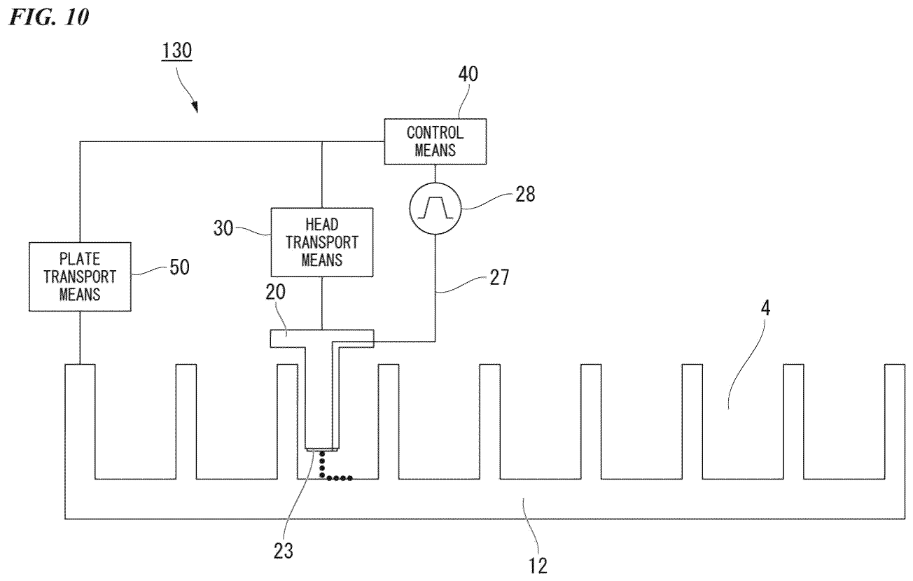

FIG. 8 is a schematic diagram showing an example of a cell culture substrate manufacturing apparatus. FIG. 9 and FIG. 10 are schematic diagrams showing other modified examples of the cell culture substrate manufacturing apparatus of FIG. 8.

As shown in FIG. 8, a cell culture substrate manufacturing apparatus 110 has an ejecting head (droplet ejecting means) 20, a head transport means 30, and a control means 40. In addition, in the cell culture substrate manufacturing apparatus 110, a driving waveform supply source 28 for the piezoelectric element is connected to the piezoelectric element of the ejecting head 20 via the wiring 27.

In the cell culture substrate manufacturing apparatus 110 shown in FIG. 8, changing the relative position of the ejecting head 20 and the well plate 12 by the head transport means 30 connected to the control means 40 in synchronization with the ejecting of the droplets makes it possible to arrange the droplets of the second liquid on the bottom surface of the well 4 at any position.

For the means for changing the relative position of the ejecting head 20 and the well plate 12, a transport means may be arranged only in the ejecting head 20 as shown in FIG. 8, a transport means may be arranged only in the well plate 12 as shown in FIG. 9, or a transport means may be arranged in both the ejecting head 20 and the well plate 12 as shown in FIG. 10.

For example, in the cell culture substrate manufacturing apparatus 130 shown in FIG. 10, it is also possible to control the operation in the left-right direction of the paper surface using the head transport means 30 and to control the operation in the front-back direction of the paper surface using the plate transport means 50.

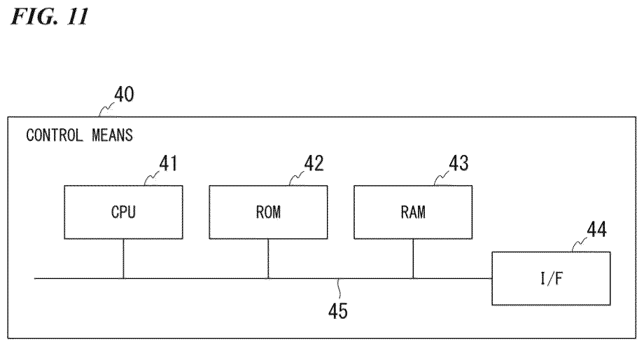

The control means 40 has the function of controlling the driving waveform supply source 28, the head transport means 30, and the plate transport means 50. Referring to FIG. 11, a description will be given below of the operation of the cell culture substrate manufacturing apparatus containing the operation of the control means 40.

FIG. 11 is a diagram illustrating the hardware block of the control means 40 of FIG. 8 to FIG. 10.

As shown in FIG. 11, the control means 40 has a CPU 41, a ROM 42, a RAM 43, an I/F 44, and a bus line 45. The CPU 41, the ROM 42, the RAM 43, and the I/F 44 are connected to each other via the bus line 45.

The CPU 41 controls each function of the control means 40.

The ROM 42, which is a storage means, stores programs that are executed by the CPU 41 to control each function of the control means 40 and various types of information.

The RAM 43, which is a storage means, is used as a work area or the like for the CPU 41. In addition, it is possible for the RAM 43 to temporarily store predetermined information.

The I/F 44 is an interface for connecting the cell culture substrate manufacturing apparatus to other devices and the like. The cell culture substrate manufacturing apparatus may be connected to an external network or the like via the I/F 44.

[Second Liquid]

The second liquid is a suspension including a second fixative and cells.

Examples of the cells are the same as exemplified in the cell culture substrate according to the first embodiment described above.

(Second Fixative)

The second fixative is not particularly limited as long as the second fixative is mixed with the first fixative to form a cross-linked structure and is able to be selected for the purpose as appropriate. Specific examples of the second fixative include polysaccharides, polyvalent metal salts, fibrinogen, thrombin, fibronectin, laminin, recombinant peptides, chitosan, chitin, tetrafunctional polyethylene glycol (Tetra-PEG), and the like. In addition, it is also possible to use the above examples modified with functional groups such as maleimidyl groups. The above may be used alone or may be used in a combination of two or more types. Among the above, Tetra-PEG-maleimidyl is preferable in a case where the first fixative is thiol gelatin.

(Other Components)

The second liquid may further include other components as necessary.

The other components are not particularly limited and are able to be selected for the purpose as appropriate and examples thereof include culture media, cross-linking agents, pH adjusters, preservatives, antioxidants, and the like.

It is possible to prepare the second liquid by adding the cells, the second fixative, and, as necessary, other components to a buffer solution such as water or phosphate buffered saline, or to a culture medium to a desired concentration, and carrying out mixing.

[Hydrogel]

The hydrogel has a polymer formed by bonding the first fixative and second fixative, and water included in the polymer.

In one-component gels, since it takes time for the gel to cure, when the culture medium is filled quickly, the hydrogel particles do not adhere to the bottom surface of the substrate and are washed away. In contrast, the hydrogel formed by the method for manufacturing a cell culture substrate of the present embodiment is a two-component gel which is formed by reacting at least a part of the first liquid, which forms the film, and the second liquid, which forms the droplets 9a. Therefore, the curing time of the gel is shorter than that of a one-component gel and it is possible to cure the gel to the desired hardness by the time the falling droplets arrive at the bottom surface of the substrate.

For example, in a case where the first fixative is Tetra-PEG-maleimidyl and the second fixative is thiol gelatin, a thioether group is formed by the reaction of the maleimidyl group and thiol group and it is possible to use a hydrogel having a Tetra-PEG-constituent unit, a polymer having a gelatin-constituent unit, and water included in the polymer.

<Culture Medium Adding Step>

In addition to the above film forming step and the above droplet ejecting step, the method for manufacturing a cell culture substrate of the present embodiment may further include a step for adding a culture medium to the hydrogel particles after the cells are fixed to the substrate via the hydrogel (may be referred to below as a “culture medium adding step”).

The method of adding the culture medium is not particularly limited and is able to be selected for the purpose as appropriate and examples thereof include a pipette dispensing method using a micropipette or the like, a micromanipulator method, an aspirator method, an ink jet method, transfer methods such as a screen-printing method, and the like.

[Culture Medium]

The culture medium is a solution that includes the components necessary for the formation and maintenance of tissue bodies, prevents drying, and adjusts an external environment having osmotic pressure or the like and is able to be selected as appropriate from known culture media in the field. The culture medium may be removed as appropriate in a case where constant immersion in a culture medium solution is not necessary.

The culture medium is not particularly limited and is able to be selected for the purpose as appropriate and examples thereof include culture media classified by composition such as natural culture media, semi-synthetic culture media, and synthetic culture media; culture media classified by shape such as semi-solid culture media, liquid culture media, and powdered culture media (may also be referred to below as “powder culture media”), and the like. The above may be used alone or may be used in a combination of two or more types. In a case where the cells are derived from animals, it is possible to use any culture media used for culturing animal cells.

The culture medium used for culturing animal cells is not particularly limited and is able to be selected for the purpose as appropriate and examples thereof include Dulbecco's Modified Eagle Medium (D-MEM), Ham's F12 medium (Ham's Nutrient Mixture F12), D-MEM/F12 medium, McCoy's 5A medium, Eagle's Minimum Essential Medium (E-MEM), a MEM (alpha Modified Eagle's Minimum Essential Medium), MEM (Minimum Essential Medium), RPMI 1640 medium, Iscove's Modified Dulbecco's Medium (I-MDM), MCDB 131 medium, William's medium E, IPL 41 medium, Fischer's medium, StemPro34 (manufactured by Invitrogen), X-VIVO 10 (manufactured by Cambrex Corporation), X-VIVO 15 (manufactured by Cambrex Corporation), HPGM (manufactured by Cambrex Corporation), StemSpan H3000 (manufactured by Stemcell Technologies), StemSpan SFEM (manufactured by Stemcell Technologies), StemlineII (manufactured by Sigma-Aldrich), QBSF-60 (manufactured by Quality Biological), StemProhESCSFM (manufactured by Invitrogen), Essential8 (registered trademark) medium (manufactured by Gibco), mTeSR1 or 2 medium (manufactured by Stemcell Technologies), ReproFF or ReproFF2 (manufactured by Reprocell), PSGro hESC/iPSC medium (manufactured by Systems Biosciences), NutriStem (registered trademark) medium (manufactured by Biological Industries), CSTI-7 medium (manufactured by Cell Science & Technology Institute, Inc.), MesenPRO RS medium (manufactured by Gibco), MF-Medium (registered trademark) mesenchymal stem cells growth medium (manufactured by Toyobo Co., Ltd.), Sf-900II (manufactured by Invitrogen), Opti-Pro (manufactured by Invitrogen), and the like. The above may be used alone or may be used in a combination of two or more types.

The carbon dioxide concentration in the culture medium is not particularly limited and is able to be selected for the purpose as appropriate, but 2 v/v % or more and v/v % or less is preferable and 3 v/v % or more and 4 v/v % or less is more preferable. When the carbon dioxide concentration is in the range described above, it is possible to suitably culture the cells.

FIG. 12 is a schematic diagram showing processes of the method for manufacturing a cell culture substrate according to one embodiment of the present invention. With reference to FIG. 12, a description will be given of the method for manufacturing a cell culture substrate of the present embodiment.

As shown in the film forming step in FIG. 12, the first liquid is added to the interior of the recess 2 formed at the bottom surface of the well 4 using a micropipette 7, such that the film thickness of the first liquid is equal to the depth of the recess 2. Next, as shown in the droplet ejecting step in FIG. 12, using the ejecting head 20, the second liquid 9 is ejected as the droplets 9a by the ink jet method to contact the film 6 of the first liquid formed in the interior of the recess 2. By bringing the droplets 9a of the second liquid into contact with the film 6 of the first liquid, the first fixative and second fixative react to form the particles 3 of hydrogel. It is possible to produce a cell culture substrate in which cells are adhered to the substrate via the formed hydrogel. Next, it is possible to prevent drying of the hydrogel particles and to prepare the hydrogel particles for evaluation by dropping a culture medium 10 onto the hydrogel particles 3 using the micropipette 7 as shown in the culture medium adding step in FIG. 12.

The cell culture substrate obtained by the method for manufacturing a cell culture substrate of the present embodiment is suitably used for the evaluation of the toxicity or drug efficacy of a test substance.

In a case where the cell culture substrate of the present embodiment is used for the evaluation of the toxicity or drug efficacy of a test substance, it is possible to perform the evaluation by using the culture medium 10 shown in the culture medium adding step in FIG. 12, to which the test substance to be evaluated for toxicity or drug efficacy is added beforehand.

The test substance to be evaluated for toxicity is not particularly limited and is able to be selected for the purpose as appropriate. Specific examples thereof include zinc chloride, 1-butanol, benzoic acid, ethyl vanillin, 4-hydroxybenzoic acid, sulfanilic acid, tartaric acid, methyl salicylate, salicylic acid, sodium lauryl sulfate, lactic acid, benzyl alcohol, dextran, diethyl phthalate, glycerol, propyl paraben, Tween 80, dimethyl isophthalate, phenol, chlorobenzene, sulfanilamide, octanoic acid, and the like.

The test substance to be evaluated for drug efficacy is not particularly limited and is able to be selected for the purpose as appropriate. Specific examples thereof include oxazolone, benzoquinone, 2,4-dinitrochlorobenzene, 4-phenylenediamine, glutaraldehyde, benzoyl peroxide, 4-methylaminophenol sulfate, formaldehyde, cinnamaldehyde, ethylenediamine, 2-hydroxyethyl acrylate, isoeugenol, nickel (II) sulfate, benzylideneacetone, methyl 2-noninate, benzyl salutylate, diethylenetriamine, thioglycerol, 2-mercaptobenzothiazole, phenylacetaldehyde, hexyl cinnamaldehyde, dihydroeugenol, benzoisothiazolione, citral, resorcinol, phenyl benzoate, eugenol, abietic acid, amino ethyl benzoate, benzyl cinnamate, cinnamyl alcohol, hydroxycitronellal, imidazolidinyl urea, butyl glycidyl ether, ethylene glycol dimethacrylate, glyoxal, 4-nitrobenzyl bromide, and the like.

### EXAMPLES

A description will be given of the present invention using Examples, but the present invention is not limited to the following Examples.

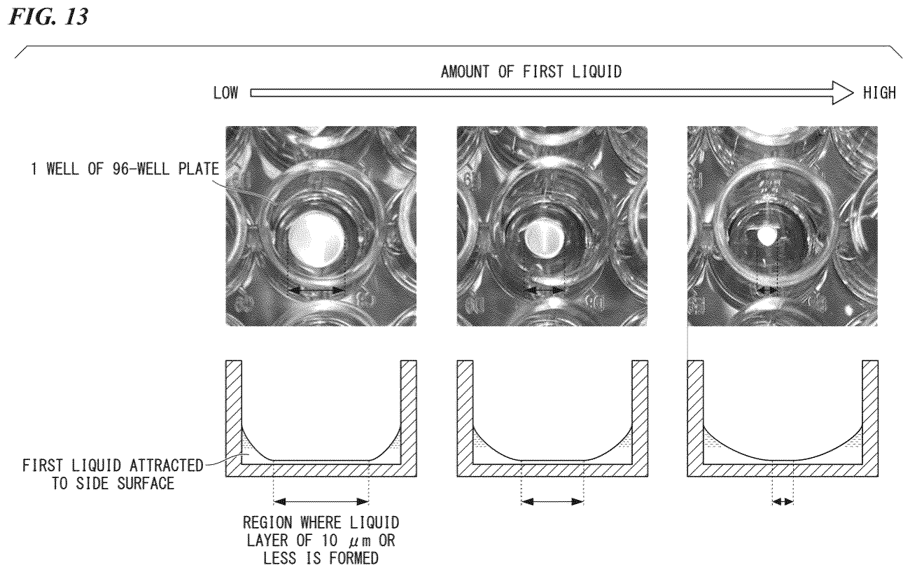

In methods of the related art such as in Patent Document 1, the first liquid is dropped onto a cell culture substrate such as a well or dish to form a film of the first liquid. At this time, as shown in FIG. 13, in a case of dropping the first liquid on a 96-well plate, it was difficult to form a uniform thin film of the first liquid with the desired thickness in the wells because the first liquid was attracted to both the corners and side surfaces of the well plate due to surface tension. In addition, the thin film in the vicinity of the center of the well formed by this method is 10 μm or less such that it is not possible to form hydrogel particles approximately 100 μm in diameter.

Therefore, it was hypothesized that by forming recesses in the cell culture substrate, the depth of the recesses would act as a barrier stopping the first liquid being attracted to the corners and wall surfaces of the well plate and make it possible to form a uniform thin film of the first liquid with the desired thickness. In the present Example, as an example of a method for forming a recess, forming a recess on a cell culture substrate by arranging a ring of silicone rubber in a well was examined.

<Materials>

[Preparation of First Liquid]

To 1 mL of phosphate buffered saline (manufactured by Life Technologies, Inc.; may be referred to below as “PBS (-)”), 50 mg of thiol gelatin (manufactured by Sigma-Aldrich, trade name “Thiol functionalized gelatin”) was added as the first fixative to prepare a 50 mg/mL thiol gelatin aqueous solution and obtain the first liquid.

[Preparation of Second Liquid]

1×10⁵ NIH/3T3 cells (JCRB0615, available from JCRB Cell Bank; may be simply referred to below as “3T3 cells”), which are a mouse embryonic fibroblast cell line, were suspended in 30 mL of Dulbecco's Modified Eagle Medium (manufactured by Life Technologies, Inc., trade name “DMEM(1x); may be referred to below as “D-MEM”) and the obtained cell suspension was added to a 100 mm dish and cultured for 72 hours in an incubator (manufactured by Panasonic Corporation, apparatus name “KM-CC17RU2”, 37° C., 5% CO₂ environment). After culturing, the culture solution was removed and washed by adding 2 mL of Dulbecco's phosphate buffered saline (may be referred to below as “DPBS”). The DPBS was removed, 2 mL of trypsin (manufactured by Life Technologies, Inc., trade name “0.05% Trypsin-EDTA (1x)”) was added, and cells were isolated by a trypsin treatment for 5 minutes in the above incubator (37° C., 5% CO₂ environment).

10 mg of Tetra-PEG-maleimidyl (trade name “SUNBRIGHT PTE-100MA” manufactured by Yuka Sangyo Co., Ltd.) was added in advance as a second fixative to 1 mL of PBS (-) to prepare a 10 mg/mL Tetra-PEG-maleimidyl aqueous solution. Then, as described above, the isolated cells were added to the 10 mg/mL thiol gelatin aqueous solution at a concentration of 1×10 7 cells/mL to obtain a second liquid containing the second fixative and cells.

<Manufacturing of Cell Culture Substrate>

### Example 1

A silicone rubber ring (5 mm in diameter and 100 μm in thickness) was arranged in each well of a 96-well plate (manufactured by Corning, trade name: “96-well cell culture plate, flat bottom, low evaporation type, with lid, polystyrene) to obtain a cell culture substrate having recesses formed at the bottom of the wells. The silicone rubber rings were produced by subjecting a silicone rubber sheet (manufactured by Kenis Ltd., 300 mm×300 mm×100 μm) to punching processing to form ring shapes with a diameter of 5 mm and a thickness of 100 μm.

2.5 μL of a 50 mg/mL thiol gelatin aqueous solution was added into the recesses of each well of the 96-well plate using a micropipette (refer to the “film forming step” in FIG. 12). The film thickness of the first liquid was approximately 100 μm.

Next, using an ink jet method bioprinter (in-house developed product, apparatus name “cell-compatible ink jet apparatus”), the second liquid was ejected in 100 μm diameter droplets into the recesses of the wells of a 96-well plate, such that the hydrogel particles including cells were arranged without overlapping (refer to the “droplet ejecting step” in FIG. 12). Thiol gelatin, which is the first fixative, and Tetra-PEG-maleimidyl, which is the second fixative, were mixed to form particles of hydrogel and, after a few minutes of standing at room temperature, the hydrogel particles including cells were fixed to the bottom surface of the recess. The height of the hydrogel particles was approximately 40 μm and the shape thereof was uniform. After confirming that the hydrogel particles were fixed, the solution was removed and 0.1 mL of D-MEM was added instead to culture the cells (refer to the “culture medium adding step” in FIG. 12).

### COMPARATIVE EXAMPLE 1

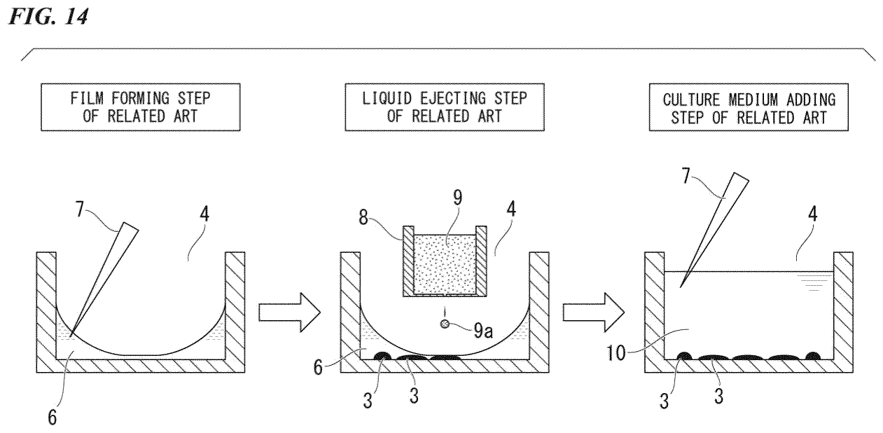

15 μL of a 50 mg/mL thiol gelatin aqueous solution was added using a micropipette to each well of a 96-well plate (manufactured by Corning, trade name: “96-well cell culture plate, flat bottom, low evaporation type, with lid, polystyrene) (refer to the “film forming step of related art” in FIG. 14).

Next, using an ink jet method bioprinter (in-house developed product, apparatus name “cell-compatible ink jet apparatus”), the second liquid was ejected in 100 μm diameter droplets onto the wells of a 96-well plate, such that the hydrogel particles including cells were arranged without overlapping (refer to the “droplet ejecting step of related art” in FIG. 14). Thiol gelatin, which is the first fixative, and Tetra-PEG-maleimidyl, which is the second fixative, were mixed to form hydrogel particles and, after a few minutes of standing at room temperature, the particles of hydrogel including cells were fixed to the bottom surface of the recess. The height of the hydrogel particles varied from approximately several μm to 10 μm and the shape thereof was non-uniform. After confirming that the hydrogel particles were fixed, the solution was removed and 0.1 mL of D-MEM was added instead to culture the cells (refer to the “culture medium adding step of related art” in FIG. 14).

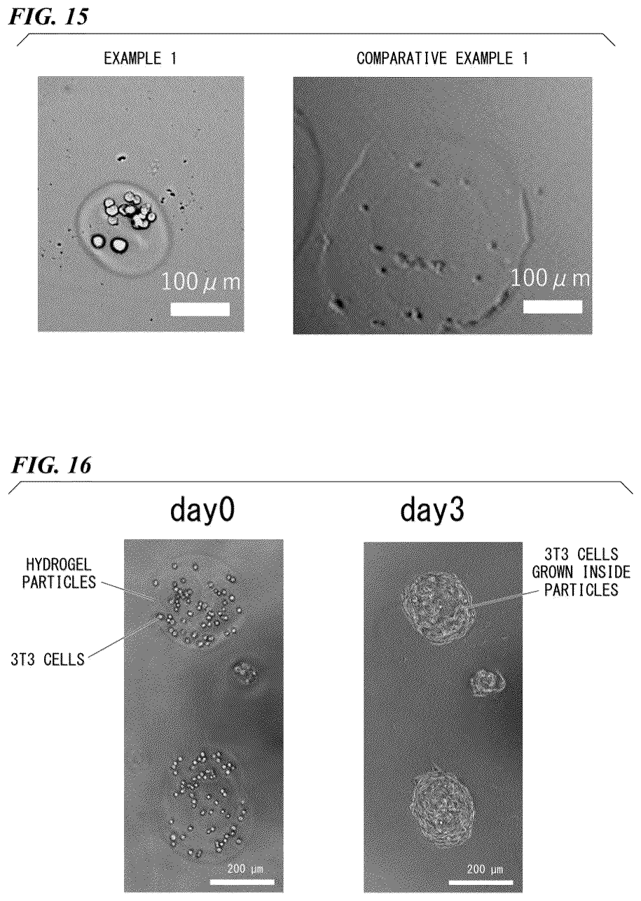

The hydrogel particles on the cell culture substrate immediately after manufacturing as obtained in Example 1 and Comparative Example 1 were observed using an optical microscope (manufactured by Olympus Corporation, apparatus name “CKX53”). The results are shown in FIG. 15.

As shown in FIG. 15, in the cell culture substrate obtained in Example 1, it was confirmed that the hydrogel particles in which cells were encapsulated were formed to have a diameter of approximately 100 μm when viewed in plan view. On the other hand, in the cell culture substrate obtained in Comparative Example 1, it was clear that the hydrogel particles had a flattened shape and that the cells were easily dried and damaged in this environment.

In addition, FIG. 16 shows microscopic images of the hydrogel particles on the cell culture substrate obtained in Example 1 immediately after manufacturing (day 0) and 72 hours thereafter (day 3).

As shown in FIG. 16, it was confirmed that, in the period from day 0 to day 3, 3T3 cells continued to grow in the hydrogel particles in a state where the hydrogel particles remained fixed to the bottom surfaces of the recesses in the wells.

From the above, it is clear that, in the method for manufacturing a cell culture substrate of the present embodiment, by forming a recess in the surface of the substrate, it is possible to uniformly form a liquid film at the desired thickness and to obtain uniformly shaped hydrogel particles including cells.

The present invention includes the following aspects.

(1) A method for manufacturing a cell culture substrate in which cells are fixed via a hydrogel on a surface of a substrate, the method including a step for forming a film of a first liquid on the surface of the substrate, and a step for ejecting droplets of a second liquid onto the film, in which the hydrogel has a polymer formed by bonding a first fixative and a second fixative, and water included in the polymer, the first liquid is an aqueous solution including the first fixative, the second liquid is a suspension including the second fixative and the cells, a recess is formed in the substrate at a position where the film is to be formed and the film fills an interior of the recess, at least parts of the first liquid forming the film and the second liquid forming the droplets are mixed to form particles of hydrogel corresponding to a size of the droplets in the step for ejecting the droplets, and the particles encapsulate the cells and are attached to the surface of the substrate.

(2) The method for manufacturing a cell culture substrate according to (1), in which a diameter of the droplets is equal to or less than a film thickness of the film as defined by a depth of the recess.

(3) The method for manufacturing a cell culture substrate according to (1) or (2), in which the recess is a portion surrounded by a convex strip provided in a ring shape on the substrate.

(4) The method for manufacturing a cell culture substrate according to (3), in which the convex strip is formed of a member removable from the substrate.

(5) The method for manufacturing a cell culture substrate according to (4), in which the member is made of a silicone rubber.

(6) The method for manufacturing a cell culture substrate according to any one of (1) to (5), in which the substrate is a well plate, and the recess is formed at a bottom of one or more wells of the well plate.

(7) The method for manufacturing a cell culture substrate according to any one of (1) to (6), in which the second liquid is ejected by an ink jet type ejecting method in the step for ejecting the droplets.

(8) A cell culture substrate including a substrate, and hydrogel particles attached to a surface of the substrate and encapsulating cells, in which a recess is formed in the surface of the substrate, the hydrogel particles are attached to a bottom surface of the recess, and a height of the hydrogel particles is less than a depth of the recess.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

EXPLANATION OF REFERENCES
1: substrate, 2: recess, 2a: bottom surface of recess, 2b: edge of recess, 3: hydrogel particles, 4: well, 5: convex strip, 5a: upper surface of convex strip, 6: film of first liquid, 7: pipette, 8: ejecting head, 9: second liquid, 9a: droplet of second liquid, 10: culture medium, 11: cells, 12: well plate, 20: ejecting head, 21: liquid chamber member, 22: elastic member, 23: MEMS chip, 24: piezoelectric element, 25: nozzle, 26: membrane, 27: wiring, 28: driving waveform supply source for piezoelectric element, 30: head transport means, 40: control means, 41: CPU, 42: ROM, 43: RAM, 44: I/F, 45: bus line, 50: plate transport means, 100, 200, 300, 400, 500, 600: cell culture substrate, 110, 120, 130: cell culture substrate manufacturing apparatus, D_{R}: depth of recess, D_{W}: depth of well, H_{C}: height of hydrogel particle

### PRIOR ART REFERENCES

### Patent Document

[Patent Document 1] Japanese Patent No. 6977347

What is claimed is:

1. A method for manufacturing a cell culture substrate in which cells are fixed via a hydrogel on a surface of a substrate, the method comprising:
forming a film of a first liquid on the surface of the substrate; and
ejecting droplets of a second liquid onto the film,
wherein the hydrogel has a polymer formed by bonding a first fixative and a second fixative, and water included in the polymer,
the first liquid is an aqueous solution including the first fixative,
the second liquid is a suspension including the second fixative and the cells,
a recess is formed in the substrate at a position where the film is to be formed and the film fills an interior of the recess,
at least parts of the first liquid forming the film and the second liquid forming the droplets are mixed to form particles of hydrogel corresponding to a size of the droplets in the ejecting, and
the particles encapsulate the cells and are attached to the surface of the substrate.

2. The method for manufacturing a cell culture substrate according to claim 1,
wherein a diameter of the droplets is equal to or less than a film thickness of the film as defined by a depth of the recess.

3. The method for manufacturing a cell culture substrate according to claim 1,
wherein the recess is a portion surrounded by a convex strip provided in a ring shape on the substrate.

4. The method for manufacturing a cell culture substrate according to claim 3,
wherein the convex strip is formed of a member removable from the substrate.

5. The method for manufacturing a cell culture substrate according to claim 4,
wherein the member is made of a silicone rubber.

6. The method for manufacturing a cell culture substrate according to claim 1,
wherein the substrate is a well plate, and
the recess is formed at a bottom of one or more wells of the well plate.

7. The method for manufacturing a cell culture substrate according to claim 1,
wherein the second liquid is ejected by an ink jet type ejecting method in the ejecting.

8. A cell culture substrate comprising:
a substrate; and
hydrogel particles attached to a surface of the substrate and encapsulating cells,
wherein a recess is formed in the surface of the substrate,
the hydrogel particles are attached to a bottom surface of the recess, and
a height of the hydrogel particles is less than a depth of the recess.
